(19) United States
(12) Patent Application Publication
(10) Pub. No.: US 2006/0153907 A1
(43) Pub. Date: Jul. 13, 2006

(54) Liposome formulations of boronic acid compounds

(76) Inventors: Samuel Zalipsky, Redwood City, CA (US); Francis J. Martin, San Francisco, CA (US)

Correspondence Address: PERKINS COIE LLP, P.O. BOX 2168, MENLO PARK, CA, 94026, US

(21) Appl. No.: 11/267,794

(22) Filed: Nov. 4, 2005

Related U.S. Application Data
(60) Provisional application No. 60/625,216, filed on Nov. 5, 2004.

(51) Int. Cl. A61K 31/69; A61K 9/127

(52) U.S. Cl. 424/450; 514/64

(57) ABSTRACT

A liposome composition comprised of liposomes having a peptide boronic acid proteasome inhibitor compound entrapped in the liposomes is described. The boronic acid compound is entrapped in the liposomes in the form of a boronate ester, subsequent to interaction with a liposome-entrapped polyol. In one embodiment, the liposomes have an outer coating of hydrophilic polymer chains and are used to treat a malignancy in a subject.

### CROSS-REFERENCE TO RELATED APPLICATIONS

This application claims the benefit of U.S. Provisional Application No. 60/625,216, filed Nov. 5, 2004, which is incorporated herein by reference in its entirety.

### TECHNICAL FIELD

The subject matter described herein relates to a liposome composition comprising a boronic acid compound, and in particular a peptide boronic acid compound in the form of a boronate ester.

### BACKGROUND

Liposomes, or lipid bilayer vesicles, are spherical vesicles comprised of concentrically ordered lipid bilayers that encapsulate an aqueous phase. Liposomes serve as a delivery vehicle for therapeutic and diagnostic agents contained in the aqueous phase or in the lipid bilayers. Delivery of drugs in liposome-entrapped form can provide a variety of advantages, depending on the drug, including, for example, a decreased drug toxicity, altered pharmacokinetics, or improved drug solubility. Liposomes when formulated to include a surface coating of hydrophilic polymer chains, so-called Stealth® or long-circulating liposomes, offer the further advantage of a long blood circulation lifetime, due in part to reduced removal of the liposomes by the mononuclear phagocyte system. Often an extended lifetime is necessary in order for the liposomes to reach their desired target region or cell from the site of injection.

Ideally, such liposomes can be prepared to include an entrapped therapeutic or diagnostic compound (i) with high loading efficiency, (ii) at a high concentration of entrapped compound, and (iii) in a stable form, i.e., with little compound leakage on storage.

Methods for forming liposomes under conditions in which the compound to be entrapped is passively loaded into the liposomes are well known. Typically, a dried lipid film is hydrated with an aqueous phase medium, to form multi-lamellar vesicles which passively entrap compound during liposome formation. The compound may be either a lipophilic compound included in the dried lipid film, or a water-soluble compound contained in the hydrating medium. For water-soluble compounds, this method gives rather poor encapsulation efficiencies, in which typically only 5-20% of the total compound in the final liposome suspension is in encapsulated form. Additional compound may be lost if the vesicles are further processed, i.e., by extrusion, to produce smaller, more uniformly sized liposomes. The poor encapsulation efficiency limits the amount of compound that can be loaded into the liposomes, and can present costly compound-recovery costs in manufacturing.

A variety of other passive entrapment methods for forming compound-loaded liposomes, including solvent injection methods and a reverse-evaporation phase approach have been proposed (Szoka, F. and Papahadjopoulos, D., Proc. Natl. Acad. Sci. USA 75:4194-4198, (1978)). These methods tend to suffer from relatively poor loading efficiencies and/or difficult solvent handling problems.

It has also been proposed to passively load compounds into liposomes by incubating the compound with preformed liposomes at an elevated temperature at which the compound is relatively soluble, allowing the compound to equilibrate into the liposomes at this temperature, then lowering the temperature of the liposomes to precipitate compound within the liposomes. This method is limited by the relatively poor encapsulation efficiencies which are characteristic of passive loading methods. Also, the compound may be quickly lost from the liposomes at elevated temperature, e.g., body temperature.

Compound loading against an inside-to-outside pH or electrochemical liposome gradient has proven useful for loading ionizable compounds into liposomes. In theory, very high loading efficiencies can be achieved by employing suitable gradients, e.g., pH gradients of 2-4 units, and by proper selection of initial loading conditions (Nichols and Deamer, D., Biochim. Biophys. Acta 455:269-171, (1976)). With this method, compound leakage from the liposomes will follow the loss of ion gradient from the liposomes. Therefore, compound can be stably retained in liposome-encapsulated form only as long as the ion gradient is maintained.

This gradient stability problem was addressed, and at least partially solved, by employing an ammonium salt gradient for compound loading (Haran, G., et al., Biochim. Biophys. Acta 1151:201-215, (1993)). Excess ammonium ions, which act as a source of protons in the liposomes, function as a battery to replenish protons lost during storage, thus increasing the lifetime of the proton gradient, and therefore reducing the rate of leakage from the liposomes. The method is limited to ionizable amine compounds.

The gradient stability problem has also been addressed by including an ionizable trapping agent in the liposomes, to serve as a counterion to the ionizable compound and to form an ionization complex and a precipitate therewith (U.S. Pat. No. 6,110,491). Another approach described in the art for loading and retaining a weakly acidic compound containing at least one carboxyl group inside liposomes is to include a cation in the liposomes that will salt out or precipitate the compound (U.S. Pat. No. 5,939,096).

U.S. Pat. No. 5,380,531 describes liposomes having an entrapped amino acid or peptide, where the C-terminus of the amino acid or peptide is modified to a non-acidic group, such as an amide or a methyl ester and the modified amino acid or peptide is loaded into the liposomes against a transmembrane ion gradient. The modified amino acid or peptide acts as a weak base and the compound is driven into the liposomes by virtue of a low internal liposome pH and a high external liposome pH gradient. The compound protenates upon reaching the internal liposome space and is retained in the liposome in protenated form.

Despite these various approaches to loading therapeutic compounds into liposomes, some compounds remain difficult to load into a liposome, particularly in a high drug to lipid ratio for clinical efficacy. One such compound is bortezomib, previously known as PS-341 (Velcade®, Millennium Pharmaceuticals, Inc, Cambridge, Mass.). Bortezomib is a dipeptide boronic acid derivative and was synthesized as a highly selective, potent, reversible proteasome inhibitor with a Kᵢ of 0.6 nmol/L (Adams, et al., Semin. Oncol., 28(6):613-619 (2001)). Using the National Cancer Institute's in vitro screen, bortezomib showed cytotoxicity against a range of tumor lines (Adams, Id.) and had antitumor activity in human prostate (Frankel et al., Clin. Cancer Res., 6(9):3719-3728 (2000); DiPaola et al., Hematol. Oncol. Clin. North Am., 15(3):509-524 (2001)) and lung cancer xenograft models (Oyaizu et al., Oncol. Rep., 8(4):825-829 (2001)).

Peptide boronic acids such as bortezomib are derivatives of usually short 2-4 amino acid peptides containing aminoboronic acid at the acidic end, C-terminal end, of the sequence (Zembower et al., Int. J. Pept. Protein Res., 47(5):405-413 (1996)). Due to the ability to form a stable tetrahedral borate complex between the boronic acid group and the active site serine or histidine moiety, peptide boronic acids are powerful serine-protease inhibitors. This activity is often enhanced and made highly specific towards a particular protease by varying the sequence of the peptide boronic acids and introducing unnatural amino acid residues and other substituents. This led to the selection of peptide boronic acids with powerful antiviral (Priestley, E. S. and Decicco, C. P., Org. Lett., 2(20):3095-3097 (2000); Bukhtiyarova, M. et al., Antivir. Chem. Chemother., 12(6):367-73 (2001); Archer, S. J. et al., Chem. Biol., 9(1):79-92 (2002); Priestley, E. S. et al., Bioorg. Med. Chem. Lett., 12(21):3199-202 (2002)) and cytotoxic activities. (Teicher, B. A. et al., Clin. Cancer Res., 5(9):2638-2645 (1999); Frankel et al., Clin. Cancer Res., 6(9):3719-3728 (2000); Lightcap, E. S. et al., Clin. Chem., 46(5):673-683 (2000); Adams, J., Semin. Oncol., 28(6):613-619 (2001); Cusack, J. C., Jr. et al., Cancer Res., 61(9):3535-3540 (2001); Shah, S. A. et al., J. Cell Biochem., 82(1):110-122 (2001); Adams, J., Curr. Opin. Chem. Biol., 6(4):493-500 (2002); Orlowski, R. Z. and Dees, E. C., Breast Cancer Res., 5(1):1-7 (2002); Orlowski, R. Z. et al., J. Clin. Oncol., 20(22):4420-4427 (2002); Schenkein, D., Clin. Lymphoma, 3(1):49-55 (2002); Ling, Y. H., et al., Clin. Cancer Res., 9(3):1145-1154 (2003)). These derivatives suffer from the same problems as other short peptides, most notably very fast clearance and inability to reach the in vivo target site.

It would be desirable to entrap such peptide boronic acid compounds into a liposomal carrier. However, there are difficulties associated with how to efficiently load these relatively non-polar dipeptides. Judging from their structures and the absence of easily ionizable amino groups, the compounds are not likely to accumulate in liposomes via pH gradient or ammonium gradient methods, discussed above. Passive encapsulation is an option, but given the non-polar nature of the compounds, it is likely they will pass through the lipid membrane with ease and thus encapsulated drug will be released with time and upon dilution.

The foregoing examples of the related art and limitations related therewith are intended to be illustrative and not exclusive. Other limitations of the related art will become apparent to those of skill in the art upon a reading of the specification and a study of the drawings.

### SUMMARY

Accordingly, in one aspect, a liposome composition comprising a peptide boronic acid compound stably entrapped in the liposomes is provided.

In another aspect, a suspension of liposomes having a peptide boronic acid compound entrapped in the liposomes in the form of a peptide boronate ester is provided.

In one aspect, the subject matter described herein relates to a composition comprising liposomes formed of a vesicle-forming lipid, and entrapped in the liposomes, a boronate ester compound prepared from a peptide boronic acid compound and a polyol.

In one embodiment, the peptide boronic acid compound is a dipeptidyl boronic acid compound, with the proviso that the dipeptidyl boronic acid compound is not bortezomib.

In another embodiment, the polyol is a compound having a cis 1,2-diol or 1,3-diol functionality. An exemplary polyol is polyvinylalcohol. Another exemplary polyol is a catecol. Other exemplary polyols are a monosaccharide, a disaccharide, an oligosaccharide, and a polysaccharide. The monosaccharide can be, for example, maltose, glucose, ribose, fructose, or sorbitol. The polyol can also be glycerol or polyglycerol or an aminopolyol, such as an aminosorbitol. In particular, copolymers of vinyl alcohol and vinyl amines are contemplated.

In another embodiment, the liposomes further comprise a higher inside/lower outside ion gradient. The ion gradient can be, for example, a hydrogen ion (pH) gradient. When the ion gradient is a pH gradient, the inside pH of the liposomes can be between about 7.5-8.5 and the pH of the environment outside the liposomes can be between about 6-7.

In another embodiment, the liposomes further include between about 1-20 mole percent of a hydrophobic moiety derivatized with a hydrophilic polymer.

In embodiments where the liposomes includes a hydrophobic moiety covalently linked to a hydrophilic polymer, a preferred polymer is polyethylene glycol. A preferred hydrophobic moiety is a lipid, and is preferably a vesicle-forming lipid.

In yet another aspect, a method of delivering a peptide boronic acid compound for treatment of a human patient is provided. The method is comprised of preparing a suspension of liposomes in an aqueous solution, the liposomes having in entrapped form, a peptidyl boronate ester compound formed from a peptide boronic acid compound and a polyol, and administering the suspension of liposomes to a subject.

In one embodiment, the liposomes are administered by injection.

In still another aspect, a method of selectively destroying tumor tissue in a tumor-bearing subject undergoing radiation therapy is disclosed. The method comprises administering to a tumor-bearing subject, liposomes having an entrapped peptide boronic acid compound covalently attached to a modified polyol to form a peptidyl boronate ester compound and an isotope of boron; and subjecting the subject to neutron-radiation therapy.

In one embodiment, the isotope of boron is in the peptide boronic acid, such as ¹⁰B.

In addition to the exemplary aspects and embodiments described above, further aspects and embodiments will become apparent by reference to the drawings and by study of the following descriptions.

### BRIEF DESCRIPTION OF THE DRAWINGS

FIGS. 1A-1P show structures of exemplary peptide boronic acid compounds;

FIG. 2 illustrates loading of an exemplary peptide boronic acid into a liposome against a higher inside/lower outside pH gradient for reaction with an entrapped polyol and formation of a boronate ester compound inside the liposome.

### DETAILED DESCRIPTION

I. Definitions

“Polyol” intends a compound having more than one hydroxyl (—OH) group.

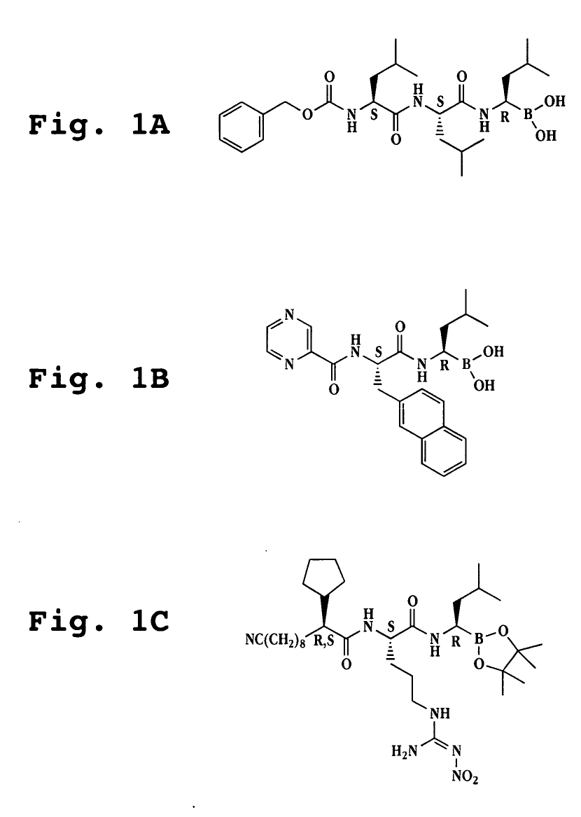

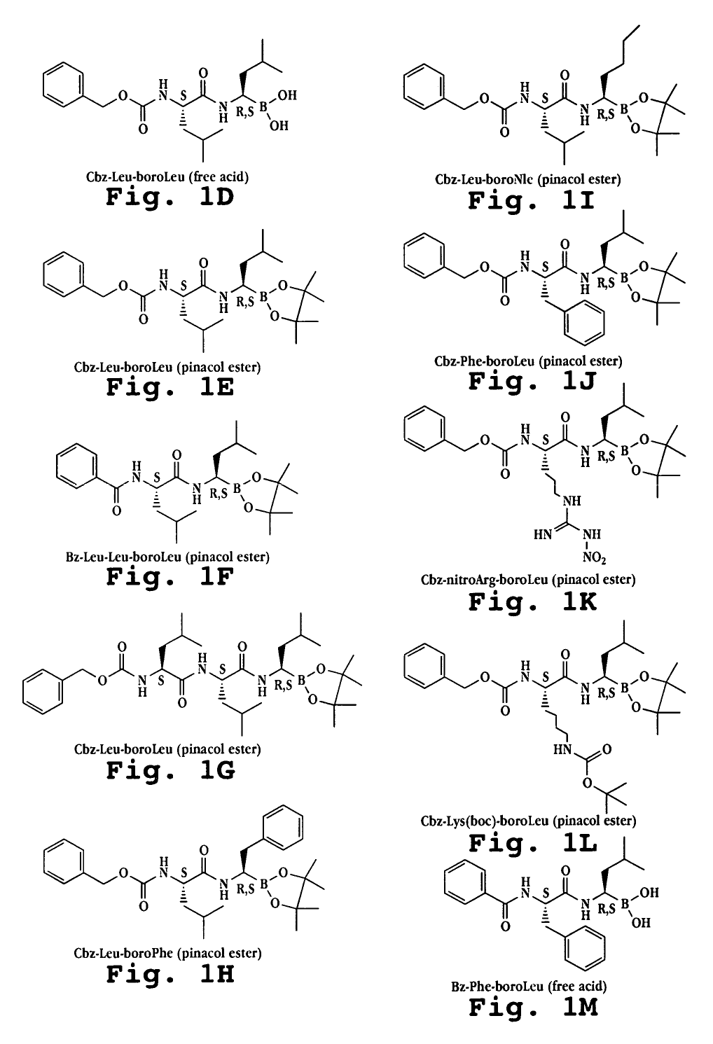

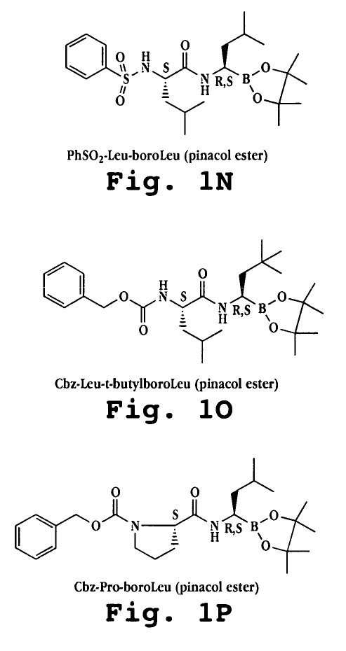

“Peptide boronic acid compound” intends a compound of the form 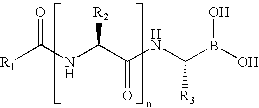
where R¹, R², and R³ are independently selected moieties that can be the same or different from each other, and n is from 1-8, preferably 1-4, with the proviso that the compound is not bortezomib (also known as Pyz-Phe-boroLeu; Pyz: 2,5-pyrazinecarboxylic acid; PS-341; Velcade®), which has the structure: 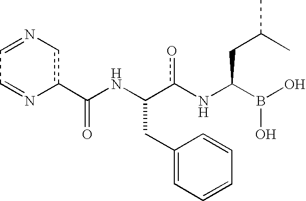
Exemplary peptide boronic acid compounds are provided in FIGS. 1A-1P.

A “hydrophilic polymer” intends a polymer having some amount of solubility in water at room temperature. Exemplary hydrophilic polymers include polyvinylpyrrolidone, polyvinylmethylether, polymethyloxazoline, polyethyloxazoline, polyhydroxypropyloxazoline, polyhydroxypropylmethacrylamide, polymethacrylamide, polydimethylacrylamide, polyhydroxypropylmethacrylate, polyhydroxyethylacrylate, hydroxymethylcellulose, hydroxyethylcellulose, polyethyleneglycol, polyaspartamide and hydrophilic peptide sequences. The polymers may be employed as homopolymers or as block or random copolymers. A preferred hydrophilic polymer chain is polyethyleneglycol (PEG), preferably as a PEG chain having a molecular weight between 500-10,000 daltons, more preferably between 750-10,000 daltons, still more preferably between 750-5000 daltons.

“Higher inside/lower outside pH gradient” refers to a transmembrane pH gradient between the interior of liposomes (higher pH) and the external medium (lower pH) in which the liposomes are suspended. Typically, the interior liposome pH is at least 1 pH unit greater than the external medium pH, and preferably 24 units greater.

“Liposome entrapped” intends refers to a compound being sequestered in the central aqueous compartment of liposomes, in the aqueous space between liposome lipid bilayers, or within the bilayer itself.

II. Liposome Formulation

In one aspect, the invention provides a liposome composition having an entrapped peptide boronic acid compound. In this section, the liposome composition and method of preparation will be described.

A. Liposome Components

As noted above, the liposome formulation is comprised of liposomes containing an entrapped peptide boronic acid compound. Peptide boronic acid compounds are peptides containing an α-aminoboronic acid at the acidic, or C-terminal, end of the peptide sequence. In general, peptide boronic acid compounds are of the form: 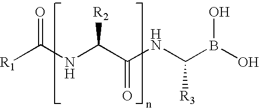
where R¹, R², and R³ are independently selected moieties that can be the same or different from each other, and n is from 1-8, preferably 1-4, with the proviso that R¹ is not 2-pyrazinyl when R² is S-benzyl and R³ is R-isobutyl. Compounds having an aspartic acid or glutamic acid residue with a boronic acid as a side chain are also contemplated.

Preferably, R¹, R², and R³ are independently selected from hydrogen, alkyl, alkoxy, aryl, aryloxy, aralkyl, aralkoxy, cycloalkyl, or heterocycle; or any of R¹, R², and R³ may form a heterocyclic ring with an adjacent nitrogen atom in the peptide backbone. Alkyl, including the alkyl component of alkoxy, aralkyl and aralkoxy, is preferably 1 to 10 carbon atoms, more preferably 1 to 6 carbon atoms, and may be linear or branched. Aryl, including the aryl component of aryloxy, aralkyl, and aralkoxy, is preferably mononuclear or binuclear (i.e. two fused rings), more preferably mononuclear, such as benzyl, benzyloxy, or phenyl. Aryl also includes heteroaryl, i.e. an aromatic ring having one or more nitrogen, oxygen, or sulfur atoms in the ring, such as furyl, pyrrole, pyridine, pyrazine, or indole. Cycloalkyl is preferably 3 to 6 carbon atoms. Heterocycle refers to a non-aromatic ring having one or more nitrogen, oxygen, or sulfur atoms in the ring, preferably a 5- to 7-membered ring having include 3 to 6 carbon atoms. Such heterocycles include, for example, pyrrolidine, piperidine, piperazine, and morpholine. Either of cycloalkyl or heterocycle may be combined with alkyl; e.g. cyclohexylmethyl.

Any of the above groups (excluding hydrogen) may be substituted with one or more substituents selected from halogen, preferably fluoro or chloro; hydroxy; lower alkyl; lower alkoxy, such as methoxy or ethoxy; keto; aldehyde; carboxylic acid, ester, amide, carbonate, or carbamate; sulfonic acid or ester; cyano; primary, secondary, or tertiary amino; nitro; amidino; and thio or alkylthio. Preferably, the group includes at most two such substituents.

Exemplary peptide boronic acid compounds are shown in FIGS. 1A-1P. Specific examples of R¹, R², and R³ shown in FIGS. 1A-1P include n-butyl and neopentyl (alkyl); phenyl or pyrazyl (aryl); 4-((t-butoxycarbonyl)amino)butyl, 3-(nitroamidino)propyl, and (1-cyclopentyl-9-cyano)nonyl (substituted alkyl); naphthylmethyl and benzyl (aralkyl); benzyloxy (aralkoxy); and pyrrolidine (R² forms a heterocyclic ring with an adjacent nitrogen atom).

In general, the peptide boronic acid compound can be a mono-peptide, di-peptide, tri-peptide, or a higher order peptide compound. Other exemplary peptide boronic acid compounds are described in U.S. Pat. Nos. 6,083,903, 6,297,217, 6,617,317, which are incorporated by reference herein.

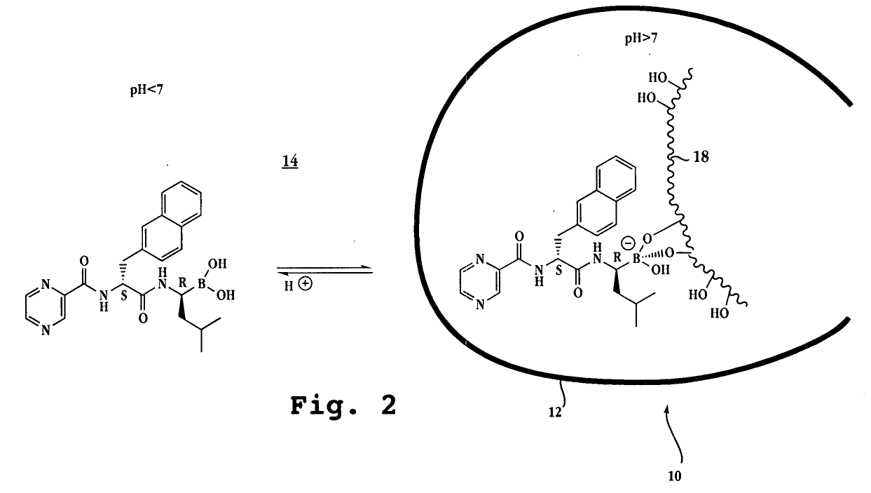

Many peptide boronic acid compounds lack an easily ionizable amino group, or are very polar, and thus are difficult to load into a liposome using conventional remote loading procedures discussed above. Thus, a loading method for peptide boronic acid compounds has been designed, to provide a liposome formulation where the peptide boronic acid compound is entrapped in the liposome in the form of a peptide boronate ester, as will now be described with respect to FIG. 2.

FIG. 2 shows a liposome 10 having a lipid bilayer membrane represented by a single solid line 12. It will be appreciated that in multilamellar liposomes the lipid bilayer membrane is comprised of multiple lipid bilayers with intervening aqueous spaces. Liposome 10 is suspended in an external medium 14, where the pH of the external medium is about 7.0 or lower, in one embodiment being less than 7.0, and in other embodiments being between about 5.5-7.0, more generally between about 6.0-7.0. Liposome 10 has an internal aqueous compartment 16 defined by the lipid bilayer membrane. Entrapped within the internal aqueous compartment is a polyol 18, examples of which are given below. The pH of the internal aqueous compartment is preferably greater than about 7.0, more preferably between about 7.1-9.0, still more preferably between about 7.5 and about 8.5.

Also entrapped in the liposome is a peptide boronic acid compound, represented in FIG. 2 by the compound of FIG. 1B, [(1R)-3-methyl-1-[[(2S)-1-oxo-3-(2-naphthyl)-2-[pyrazinylcarbonyl)amino]propyl]amino]butyl]boronic acid. It will be appreciated that the peptide boronic acid compound when entrapped in the liposome is in the form of a boronate ester compound and therefore is a modified form of the native peptide boronic acid compound, since one or more hydroxyl moieties on the native have covalently reacted with the polyol to form an ester bond. Reference herein to a peptide boronic acid compound includes the compound in native form and in modified form after reaction with a polyol. Reference herein to a polyol as a compound having more than one hydroxyl (—OH) group intends the polyol prior to reaction with a peptide boronic acid compound, since subsequent to reaction the polyol may have no remaining hydroxyl groups, one remaining hydroxyl group, or more than one hydroxyl group. A modified polyol intends a polyol having at least one hydrogen atom removed from a hydroxyl group. With continuing reference to FIG. 2, the exemplary peptide boronic acid compound is shown in the external aqueous medium, prior to passage across the lipid bilayer membrane. In the external aqueous medium, the compound is uncharged, due to the slightly acidic medium. In its uncharged state, the compound is freely permeable across the lipid bilayer. Formation of a boronate ester shifts the equilibrium to cause additional compound to permeate from the external medium across the lipid bilayer, leading to accumulation of the compound in the liposome. In another embodiment, the lower pH in the external suspension medium and the somewhat higher pH on the liposomal interior, combined with the polyol inside the liposome, induces drug accumulation into the liposome's aqueous internal compartment. Once inside the liposome, the compound reacts with the polyol to form a boronate ester. The boronate ester is essentially unable to cross the liposome bilayer, so that the drug compound, in the form of a boronate ester, accumulates inside the liposome.

The concentration of polyol inside the liposomes is preferably such that the concentration of charged groups, e.g., hydroxyl groups, is greater than the concentration of boronic acid compound. In a composition having a final drug concentration of 100 mM, for example, the internal compound concentration of the polymer charged groups will typically be at least this great.

The polyol is present at a high-internal/low-external concentration; that is, there is a concentration gradient of polyol across the liposome lipid bilayer membrane. If the polyol is present in significant amounts in the external bulk phase, the polyol reacts with the peptide boronic acid compound in the external medium, slowing accumulation of the compound inside the liposome. Thus, preferably, the liposomes are prepared, as described below, so that the composition is substantially free of polyol in the bulk phase (outside aqueous phase).

As noted above, a polyol as used herein intends a compound having more than one hydroxyl group. Monomeric and polymeric compounds containing alcoholic hydroxyl groups are contemplated. The polyol can be an aliphatic compound, a ring compound diol, a polyphenol, or the like, and examples are given below.

Non-limiting examples of monomeric polyols include sugars, glycerol, glycols, carbohydrates, amino-sugars (especially amino-sorbitol), sugar-alcohols, deoxysorbitol, gluconic acid, tartaric acid, gallic acid, etc. Simple sugars such as maltose, glucose, ribose, fructose, and sorbitol all are known to form boronate esters, with an increasing propensity for the ester formation in the listed order (Myohanen, T. A., Biochem. J., 197(3):683-688 (1981)). 1-amino-2-deoxysorbitol has an even higher tendency for boronate ester formation (Shiino, D. et al., Biomaterials, 15:121-128 (1994)). It is also contemplated that the reactivity differences among the listed sugars can be used to prepare liposome formulations with a gradient of entrapment strengths, thus fine-tuning the drug release characteristics.

Non-limiting examples of polymeric polyols include copolymers of vinyl alcohol and vinyl amine, polyethers, polyglycols, polyesters, polyalcohols, and the like. Specific examples of polymeric polyols include but are not limited to oligosaccharides, polysaccharides, polyglycerol (Hebel, A. et al., J. Org. Chem., 67(26):9452-9455 (2002)), poly(vinyl alcohol) (Kitano, S. et al., Makromol. Chem. Rapid Commun., 12:227-233 (1991)). Polyol polymers are a preferred trapping agent because upon binding of one or several drug molecules they do not tend to change their properties, such as their solubility and their ability to cross the bilayer lipid membrane.

Polyphenols as the polyol are also suitable, particularly those with an ortho diol, such as a catecol (cathechins, flavenols). In one embodiment, green tea polyphenols, alone or admixed in any combination, are contemplated for use as the polyol. At least about six cathecins are found in green tea, with (−)-epigallocatechin 3-gallate in abundance. Polyphenols from red wine are also suitable.

A preferred polyol compound is one having a plurality of cis 1,2- and/or 1,3-diol groups.

To identify a suitable polyol, a selected polyol, for example, one having a cis 1,2- and/or 1,3-diol functionality, is solubilized in a suitable solvent, typically water, at a desired concentration and at a selected pH typically around 6-8. The selected boronic acid compound is added to the solubilized polyol, at a concentration corresponding to the desired liposome-entrapped concentration. After a suitable incubation time, the mixture is inspected for formation of a boronate ester, such as by visual inspection for a precipitate or by an analytical technique. In one embodiment, formation of a precipitate of a boronate ester, exclusive of a precipitate of a weak acid salt inside the liposomes, is contemplated. This method of identifying a suitable polyol is particularly suited for identification of polymeric polyols.

The liposomes in the composition are composed primarily of vesicle-forming lipids. Such a vesicle-forming lipid is one that can form spontaneously into bilayer vesicles in water, as exemplified by the phospholipids, with its hydrophobic moiety in contact with the interior, hydrophobic region of the bilayer membrane, and its head group moiety oriented toward the exterior, polar surface of the membrane. Lipids capable of stable incorporation into lipid bilayers, such as cholesterol and its various analogues, can also be used in the liposomes. The vesicle-forming lipids are preferably lipids having two hydrocarbon chains, typically acyl chains, and a head group, either polar or nonpolar. There are a variety of synthetic vesicle-forming lipids and naturally-occurring vesicle-forming lipids, including the phospholipids, such as phosphatidylcholine, phosphatidylethanolamine, phosphatidic acid, phosphatidylinositol, and sphingomyelin, where the two hydrocarbon chains are typically between about 14-22 carbon atoms in length, and have varying degrees of unsaturation. The above-described lipids and phospholipids whose aliphatic chains have varying degrees of saturation can be obtained commercially or prepared according to published methods. Other suitable lipids include glycolipids, cerebrosides and sterols, such as cholesterol.

The vesicle-forming lipid can be selected to achieve a specified degree of fluidity or rigidity, to control the stability of the liposome in serum, and/or to control the rate of release of the entrapped agent in the liposome. Liposomes having a more rigid lipid bilayer, or a liquid crystalline bilayer, are achieved by incorporation of a relatively rigid lipid, e.g., a lipid having a relatively high phase transition temperature, e.g., up to 60° C. Rigid, i.e., saturated, lipids contribute to greater membrane rigidity in the lipid bilayer. Other lipid components, such as cholesterol, are also known to contribute to membrane rigidity in lipid bilayer structures. On the other hand, lipid fluidity is achieved by incorporation of a relatively fluid lipid, typically one having a lipid phase with a relatively low liquid to liquid-crystalline phase transition temperature, e.g., at or below room temperature.

The liposomes can optionally include a vesicle-forming lipid covalently linked to a hydrophilic polymer. As has been described, for example in U.S. Pat. No. 5,013,556, including such a polymer-derivatized lipid in the liposome composition forms a surface coating of hydrophilic polymer chains around the liposome. The surface coating of hydrophilic polymer chains is effective to increase the in vivo blood circulation lifetime of the liposomes when compared to liposomes lacking such a coating. Polymer-derivatized lipids comprised of methoxy(polyethylene glycol) (mPEG) and a phosphatidylethanolamine (e.g., dimyristoyl phosphatidylethanolamine, dipalmitoyl phosphatidylethanolamine, distearoyl phosphatidylethanolamine (DSPE), or dioleoyl phosphatidylethanolamine) can be obtained from Avanti Polar Lipids, Inc. (Alabaster, Ala.) at various mPEG molecular weights (350, 550, 750, 1000, 2000, 3000, 5000 Daltons). Lipopolymers of mPEG-ceramide can also be purchased from Avanti Polar Lipids, Inc. Preparation of lipid-polymer conjugates is also described in the literature, see U.S. Pat. Nos. 5,631,018, 6,586,001, and 5,013,556; Zalipsky, S. et al., Bioconjugate Chem., 8:111 (1997); Zalipsky, S. et al., Meth. Enzymol., 387:50 (2004). These lipopolymers can be prepared as well-defined, homogeneous materials of high purity, with minimal molecular weight dispersity (Zalipsky, S. et al., Bioconjugate Chem., 8:111 (1997); Wong, J. et al., Science, 275:820 (1997)). The lipopolymer can also be a “neutral” lipopolymer, such as a polymer-distearoyl conjugate, as described in U.S. Pat. No. 6,586,001, incorporated by reference herein.

When a lipid-polymer conjugate is included in the liposomes, typically between 1-20 mole percent of the lipid-polymer conjugate is incorporated into the total lipid mixture (see, for example, U.S. Pat. No. 5,013,556).

The liposomes can additionally include a lipopolymer modified to include a ligand, forming a lipid-polymer-ligand conjugate, also referred to herein as a ‘lipopolymer-ligand conjugate’. The ligand can be a therapeutic molecule, such as a drug or a biological molecule having activity in vivo, a diagnostic molecule, such as a contrast agent or a biological molecule, or a targeting molecule having binding affinity for a binding partner, preferably a binding partner on the surface of a cell. A preferred ligand has binding affinity for the surface of a cell and facilitates entry of the liposome into the cytoplasm of a cell via internalization. A ligand present in liposomes that include such a lipopolymer-ligand is oriented outwardly from the liposome surface, and therefore available for interaction with its cognate receptor.

Methods for attaching ligands to lipopolymers are known, where the polymer can be functionalized for subsequent reaction with a selected ligand. (U.S. Pat. No. 6,180,134; Zalipsky, S. et al., FEBS Lett., 353:71 (1994); Zalipsky, S. et al., Bioconjugate Chem., 4:296 (1993); Zalipsky, S. et al., J. Control. Rel., 39:153 (1996); Zalipsky, S. et al., Bioconjugate Chem., 8(2):111(1997); Zalipsky, S. et al., Meth. Enzymol., 387:50 (2004)). Functionalized polymer-lipid conjugates can also be obtained commercially, such as end-functionalized PEG-lipid conjugates (Avanti Polar Lipids, Inc.). The linkage between the ligand and the polymer can be a stable covalent linkage or a releasable linkage that is cleaved in response to a stimulus, such as a change in pH or presence of a reducing agent.

The ligand can be a molecule that has binding affinity for a cell receptor or for a pathogen circulating in the blood. The ligand can also be a therapeutic or diagnostic molecule, in particular molecules that when administered in free form have a short blood circulation lifetime. In one embodiment, the ligand is a biological ligand, and preferably is one having binding affinity for a cell receptor. Exemplary biological ligands are molecules having binding affinity to receptors for CD4, folate, insulin, LDL, vitamins, transferrin, asialoglycoprotein, selectins, such as E, L, and P selectins, Flk-1,2, FGF, EGF, integrins, in particular, α₄β₁αᵥβ₃, αᵥβ₁ αᵥβ₅, αᵥβ₆ integrins, HER2, and others. Preferred ligands include proteins and peptides, including antibodies and antibody fragments, such as F(ab′)₂, F(ab)₂, Fab′, Fab, Fv (fragments consisting of the variable regions of the heavy and light chains), and scFv (recombinant single chain polypeptide molecules in which light and heavy variable regions are connected by a peptide linker), and the like. The ligand can also be a small molecule peptidomimetic. It will be appreciated that a cell surface receptor, or fragment thereof, can serve as the ligand. Other exemplary targeting ligands include, but are not limited to vitamin molecules (e.g., biotin, folate, cyanocobalamine), oligopeptides, oligosaccharides. Other exemplary ligands are presented in U.S. Pat. Nos. 6,214,388; 6,316,024; 6,056,973; 6,043,094, which are herein incorporated by reference.

B. Preparation of Liposome Formulation

A peptide boronic acid compound is accumulated and trapped inside the liposomes by formation of a boronate ester between the hydroxyl functionalities on a liposome-entrapped polyol and the boronic acid compound. In brief, a polyol is disposed inside the liposomes, the peptide boronic acid compound is diffused across the liposome lipid bilayer membrane, the compound reacts with the entrapped polyol to form a boronate ester compound, thereby entrapping the compound (in modified form) in the liposome.

In one embodiment, the process is driven by pH, where a lower pH (e.g. pH 6-7) outside the liposome and somewhat higher pH (pH 7.5-8.5) on the interior of the liposome, combined with the presence of a polyol, induces accumulation and loading of the compound. In this embodiment, the composition is prepared by formulating liposomes having a higher-inside/lower-outside gradient of a polyol. An aqueous solution of the polyol, selected as described above, is prepared at a desired concentration, determined as described above. It is preferred that the polyol solution has a viscosity suitable for lipid hydration, described below. The pH of the aqueous polyol solution is preferably greater than about 7.0.

The aqueous polyol solution is used for hydration of a dried lipid film, prepared from the desired mixture of vesicle-forming lipids, non-vesicle-forming lipids (such as cholesterol, DOPE, etc.), lipopolymer, such as mPEG-DSPE, and any other desired lipid bilayer components. A dried lipid film is prepared by dissolving the selected lipids in a suitable solvent, typically a volatile organic solvent, and evaporating the solvent to leave a dried film. The lipid film is hydrated with a solution containing the polyol, adjusted to a pH of greater than about 7.0, to form liposomes.

Example 1 describes preparation liposomes composed of the lipids egg phosphatidycholine (PC), cholesterol (CHOL) and polyethylene glycol derivatized distearolphosphatidyl ethanolamine (PEG-DSPE). The lipids, at a molar ratio of 10:5:1 PC:CHOL:PEG-DSPE are dissolved in chloroform and the solvent is evaporated to form a lipid film. The lipid film is hydrated with an aqueous solution of polyvinyl alcohol, pH 7.5, to form liposomes having the polyol entrapped inside.

After liposome formation, the liposomes can be sized to obtain a population of liposomes having a substantially homogeneous size range, typically between about 0.01 to 0.5 microns, more preferably between 0.03-0.40 microns. One effective sizing method for REVs and MLVs involves extruding an aqueous suspension of the liposomes through a series of polycarbonate membranes having a selected uniform pore size in the range of 0.03 to 0.2 micron, typically 0.05, 0.08, 0.1, or 0.2 microns. The pore size of the membrane corresponds roughly to the largest sizes of liposomes produced by extrusion through that membrane, particularly where the preparation is extruded two or more times through the same membrane. Homogenization methods are also useful for down-sizing liposomes to sizes of 100 nm or less (Martin, F. J., in SPECIALIZED DRUG DELIVERY SYSTEMS—MANUFACTURING AND PRODUCTION TECHNOLOGY, P. Tyle, Ed., Marcel Dekker, New York, pp. 267-316 (1990)).

After sizing, unencapsulated bulk phase polyol is removed by a suitable technique, such as diafiltration, dialysis, centrifugation, size exclusion chromatography, or ion exchange, to achieve a suspension of liposomes having a high concentration of polyol inside and preferably little to no polyol outside. Also after liposome formation, the external phase of the liposomes is adjusted, by titration, dialysis or the like, to a pH of less than about 7.0.

The peptide boronic acid compound to be entrapped is then added to the liposome dispersion for active loading into the liposomes. The amount of peptide boronic acid compound added may be determined from the total amount of drug to be encapsulated, assuming 100% encapsulation efficiency, i.e., where all of the added compound is eventually loaded into liposomes in the form of boronate ester.

The mixture of the compound and liposome dispersion are incubated under conditions that allow uptake of the compound by the liposomes to a compound concentration that is several times that of the compound in the bulk medium, as evidence by the formation of precipitate in the liposomes. The latter may be confirmed, for example, by standard electron microscopy or x-ray diffraction techniques. Typically, the incubating is carried out at an elevated temperature, and preferably at or above the main phase transition temperature Tₘ of the liposome lipids. For high-phase transition lipids having a Tₘ of 55° C., for example, incubation may be carried out at between about 55-70° C., more preferably between about 60-70° C. The incubation time may vary from between an hour or less to up to 12 hours or more, depending on incubation temperature.

At the end of this incubation step, the suspension may be further treated to remove free (non-encapsulated) compound, e.g., using any of the methods mentioned above for removing free polymer from the initial liposome dispersion containing entrapped polyol.

Example 2 describes a method of preparing liposomes comprising a boronic acid compound and a polyol in the form of a boronate ester, where the polyol is sorbitol. In this example, a thin lipid film of egg PC and cholesterol is prepared. The lipid film is hydrated with a solution of sorbitol to form liposomes having sorbitol entrapped in the internal aqueous compartment. Unentrapped sorbitol is removed by a suitable technique, such as dialysis, centrifugation, size exclusion chromatography, or ion exchange, to achieve a suspension of liposomes having a high concentration of polyol inside and preferably little to no polyol outside. Then, the desired peptide boronic acid compound is added to the external medium. The compound in its unionized state is freely permeable across the liposomal lipid bilayers. Once inside the liposomes, the compound reacts with the entrapped polyol to form a boronate ester, shifting the equilibrium toward passage of more drug across the lipid bilayer. In this way, the peptide boronic acid compound accumulates in the liposomes and in stably entrapped therein.

Liposome formulations that include a lipid-polymer-ligand targeting conjugate can be prepared by various approaches. One approach involves preparation of lipid vesicles that include an end-functionalized lipid-polymer derivative; that is, a lipid-polymer conjugate where the free polymer end is reactive or “activated” (see, for example, U.S. Pat. Nos. 6,326,353 and 6,132,763). Such an activated conjugate is included in the liposome composition and the activated polymer ends are reacted with a targeting ligand after liposome formation. In another approach, the lipid-polymer-ligand conjugate is included in the lipid composition at the time of liposome formation (see, for example, U.S. Pat. Nos. 6,224,903; 5,620,689). In yet another approach, a micellar solution of the lipid-polymer-ligand conjugate is incubated with a suspension of liposomes and the lipid-polymer-ligand conjugate is inserted into the pre-formed liposomes (see, for example, U.S. Pat. Nos. 6,056,973; 6,316,024).

III. Methods of Use

The liposome formulation having a peptide boronic acid compound entrapped in the form of a boronate ester are used, in one embodiment, for treatment of tumor-bearing patients. In embodiments where the peptide boronic acid compound includes an isotope of boron, the liposome formulation can be used for boron neutron capture therapy. These exemplary uses will now be described.

A. Tumor Treatment

Boronic acid compounds are in the class of drugs referred to as proteasome inhibitors. Proteasome inhibitors induce apoptosis of cells by their ability to inhibit cellular proteasome activity. More specifically, in eukaryotic cells, the ubiquitin-proteasome pathway is the central pathway for protein degradation of intracellular proteins. Proteins are initially targeted for proteolysis by the attachment of a polyubiquitin chain, and then rapidly degraded to small peptides by the proteasome and the ubiquitin is released and recycled. This co-ordinated proteolytic pathway is dependent upon the synergistic activity of the ubiquitin-conjugating system and the 26S proteasome. The 26S proteasome is a large (1500-2000 kDa) multi-subunit complex present in the nucleus and cytoplasm of eukaryotes. The catalytic core of this complex, referred to as the 20S proteasome, is a cylindrical structure consisting of four heptameric rings containing α- and β-subunits. The proteasome is a threonine protease, the N-terminal threonine of the β-subunit providing the nucleophile that attacks the carbonyl group of the peptide bond in target proteins. At least three distinct proteolytic activities are associated with the proteasome: chymotryptic, tryptic and peptidylglutamyl. The ability to recognize and bind polyubiquitinated substrates is conferred by 19S (PA700) subunits, which bind to each end of the 20S proteasome. These accessory subunits unfold substrates and feed them into the 20S catalytic complex, whilst removing the attached ubiquitin molecules. Both the assembly of the 26S proteasome and the degradation of protein substrates are ATP-dependent (Almond, Leukemia, 16:433 (2002)).

The ubiquitin-proteasome system regulates many cellular processes by the coordinated and temporal degradation of proteins. By controlling levels of many key cellular proteins, the proteasome acts as a regulator of cell growth and apoptosis and disruption of its activity has profound effects on the cell cycle. For example, defective apoptosis is involved in the pathogenesis of several diseases including certain cancers, such as B cell chronic lymphocytic leukemia, where there is an accumulation of quiescent tumor cells.

Proteasome inhibitors as a class of compounds in general act by inhibiting protein degradation by the proteasome. The class includes peptide aldehydes, peptide vinyl sulfones, which act by binding to and directly inhibiting active sites within the 20S core of the proteasome. Peptide aldehydes and peptide vinyl sulfones, however, bind to the 20S core particle in an irreversible manner, such that proteolytic activity cannot be restored upon their removal. In contrast, peptide boronic acid compounds confer stable inhibition of the proteasome, yet dissociates slowly from the proteasome. The peptide boronic acid compounds are more potent than their peptide aldehyde analogs, and act more specifically in that the weak interaction between boron and sulfur means that peptide boronates do not inhibit thiol proteases (Richardson, P. G. et al., Cancer Control., 10(5):361 (2003)).

Exposure of a variety of tumor-derived cell lines to proteasome inhibitors triggers apoptosis, likely as a result of effects on several pathways, including cell cycle regulatory proteins, p53, and nuclear factor kappa B (NF-κB) (Grimm, L. M. and Osborne, B. A., Results Probl. Cell Differ., 23:209-228 (1999); Orlowski, R. Z., Cell Death Differ., 6(4):303-313 (1999)). Many of the initial studies documenting proteasome inhibitor-mediated apoptosis used cells of hematopoietic origin, including monoblasts (Imajoh-Ohmi, S. et al., Biochem. Biophys. Res. Commun., 217(3):1070-1077 (1995)), T-cell and lymphocytic leukemia cells (Shinohara, K. et al., Biochem. J., 317(Pt 2):385-388 (1996)), lymphoma cells (Tanimoto, Y. et al., J. Biochem. (Tokyo), 121(3):542-549 (1997)), and promyelocytic leukemia cells (Drexler, H. C., Proc. Natl. Acad. Sci. U.S.A., 94(3):855-860 (1997)). The first demonstration of in vivo antitumor activity of a proteasome inhibitor used a human lymphoma xenograft model (Orlowski, R. Z. et al., Cancer Res., 58(19):4342-4348 (1998)). Furthermore, proteasome inhibitors were reported to induce preferential apoptosis of patient-derived lymphoma (Orlowski, R. et al. Cancer. Res., 58:(19):4342 (1998)) and leukemia cells (Masdehors, P. et al., Br J Haematol 105(3):752-757 (1999)) and to preferentially inhibit proliferation of multiple myeloma cells (Hideshima, T. et al., Cancer Res., 61(7): 3071-3076 (2001)) with relative sparing of control, non-transformed cells. Thus, proteasome inhibitors are particularly useful as therapeutic agents in patients with refractory hematologic malignancies.

In one embodiment, a liposome formulation comprising a peptide boronic acid compound is used for treatment of cancer, and more particularly for treatment of a tumor in a cancer patient.

Multiple myeloma is an incurable malignancy that is diagnosed in approximately 15,000 people in the United States each year (Richardson, P. G. et al., Cancer Control. 10(5):361 (2003)). It is a hematologic malignancy typically characterized by the accumulation of clonal plasma cells at multiple sites in the bone marrow. The majority of patients respond to initial treatment with chemotherapy and radiation, however most eventually relapse due to the proliferation of resistant tumor cells. In one embodiment, a method for treating multiple myeloma is provided, where a liposome formulation comprising a peptide boronic acid compound entrapped in the form a boronate ester is administered to a subject suffering from multiple myeloma.

The liposome formulation is also effective in breast cancer treatment by helping to overcome some of the major pathways by which cancer cells resist the action of chemotherapy. For example, signaling through NF-κB, a regulator of apoptosis, and the p44/42 mitogen-activated protein kinase pathway, can be anti-apoptotic. Since proteasome inhibitors block these pathways, the compounds are able to activate apoptosis. Thus, a method for treating a subject having breast cancer is provided, by administering liposomes comprising a peptide boronic acid compound entrapped in the liposomes in the form of a boronate ester. Moreover, since chemotherapeutic agents such as taxanes and anthracyclines have been shown to activate one or both of these pathways, use of a proteasome inhibitor in combination with conventional chemotherapeutic agents acts to enhance the antitumor activity of drugs, such as paclitaxel and doxorubicin. Thus, in another embodiment, a treatment method is provided, where a chemotherapeutic agent, in free form or in liposome-entrapped form, is administered in combination with a liposome-entrapped peptide boronic acid compound (entrapped in the liposomes in modified form).

Doses and a dosing regimen for the liposome formulation will depend on the cancer being treated, the stage of the cancer, the size and health of the patient, and other factors readily apparent to an attending medical caregiver. Moreover, clinical studies with the proteosome inhibitor bortezomib, Pyz-Phe-boroLeu (PS-341), provide ample guidance for suitable dosages and dosing regimens. For example, given intravenously once or twice weekly, the maximum tolerated dose in patients with solid tumors was 1.3 mg/m² (Orlowski, R. Z. et al., Breast Cancer Res., 5:1-7 (2003)). In another study, bortezomib given as an intravenous bolus on days 1, 4, 8, and 11 of a 3-week cycle suggested a maximum tolerated dose of 1.56 mg/m² (Vorhees, P. M. et al., Clinical Cancer Res., 9:6316 (2003)).

The liposome formulation is typically administered parenterally, with intravenous administration preferred. It will be appreciated that the formulation can include any necessary or desirable pharmaceutical excipients to facilitate delivery.

B. Boron Neutron Capture Therapy

In another aspect, a method of administering a boron-10 isotope to a tumor, for boron-neutron capture therapy (¹⁰B-NCT), is provided. Neutron-capture therapy for cancer treatment is based on the interaction of ¹⁰B isotope with thermal neutron, each relatively innocuous, according to the following equation:
¹⁰B+¹n→⁷Li+⁴He+2.4 MeV
The reaction results in intense ionizing radiation that is confined to single or adjacent cancer cells. Thus, for successful treatment, it is desirable to deliver adequate amounts of a boron-10 isotope to tumors. The liposome formulation described herein provides a means to entrap a peptide boronic acid compound bearing a ¹⁰B isotope in a liposome. The peptide boronic acid compound bearing a ¹⁰B isotope is entrapped in the liposomes in modified form, typically as a peptide boronate, as discussed above. Liposomes that include a surface coating a hydrophilic polymer chains accumulate preferentially in tumors, due to the long blood circulation lifetime of such liposomes (see, U.S. Pat. Nos. 5,013,556; 5,213,804). The liposomes loaded with a peptide boronic acid compound bearing a ¹⁰B isotope eradicate tumors by two independent mechanisms: the liposomes act as a drug reservoir in the tumor and gradually liberate the anti-cancer compound in the tumor and the liposomes serve to accumulate sizable amounts of boron-10 isotope in the tumor assisting the efficacy of boron neutron capture therapy.

From the foregoing, the various aspects and features of the contemplated subject matter are apparent. Liposomes comprising a water-soluble, lipid bilayer impermeable polyol compound associated with a peptide boronic acid compound, to form a boronate ester, are described. The liposomes are prepared, for example, by encapsulating the polyol in the internal aqueous compartments of liposomes, removing any unencapsulated polyol from the external medium, adding the lipid bilayer permeable boronic acid compound, which passes through the lipid bilayer membrane to form a reversible ester bond with the hydroxyl moieties on the polyol. In this way, boronic acid compound, which is normally freely permeable across the lipid bilayer, is stably entrapped in the liposomes in the form of a boronate ester compound. Accumulation of the peptide boronic acid compound into the liposomes occurs in the absence of an ion gradient, however, an ion gradient can be present if desired.

IV. Examples

The following examples further illustrate the invention described herein and are in no way intended to limit the scope of the invention.

### EXAMPLE 1

### Liposomes Loaded with Peptide Boronic Acid Compound

Polyvinyl alcohol (molecular weight 2,000; Aldrich Corporation, Milwaukee, Wis.) is dissolved in water and adjusted to pH 7.4 with concentrated polyvinyl alcohol solution. A mixture of egg phosphatidyl choline, cholesterol, and polyethylene glycol-distearoylphosphatidylethanolamine (PEG-DSPE, PEG molecular weight 2,000 Da, Avanti Polar Lipids, Birmingham, Ala.) in a molar ratio of 10:5:1 is dissolved in chloroform, the solvent is evaporated in vacuum, the lipid film is incubated with shaking in the polyvinyl alcohol solution, and the lipid dispersion is extruded under pressure through 2 stacked Nucleopore® (Pleasanton, Calif.) membranes with pore size 0.2 μm. The outer buffer is exchanged for NaCl 0.14 M containing 5 mM of sodium hydroxyethylpiperazine-ethane sulfonate (HEPES) at pH 6.5 using gel chromatography on Sepharose CL-4B (Pharmacia, Piscataway, N.J.); at the same time, unentrapped polyvinyl alcohol is removed. To the so obtained liposomes, the dipeptide boronic acid compound of FIG. 1B, [(1R)-3-methyl-1-[[(2S)-1-oxo-3-(2-naphthyl)-2-[pyrazinylcarbonyl)amino]propyl]amino]butyl]boronic acid, is added. The mixture is incubated overnight at 37° C. with shaking, treated with Dowex® 50W×4 (Sigma Chemical Co., St. Louis, Mo.), and equilibrated with NaCl-HEPES solution to remove non-encapsulated bortozemib. The resulting liposomes are sterilized by filtration through a 0.2 μm filter.

### EXAMPLE 2

### Liposomes Loaded with Peptide Boronic Acid Compound

Sorbitol is dissolved in water and the pH is adjusted to 7.4. A mixture of egg phosphatidyl choline, cholesterol, and polyethylene glycol-distearoylphosphatidylethanolamine (PEG-DSPE, PEG molecular weight 2,000 Da) in a molar ratio of 10:5:1 is dissolved in chloroform and the solvent is evaporated under a vacuum. The lipid film is hydrated with the sorbitol solution and incubated with shaking to form liposome. The liposomes are extruded under pressure through 2 stacked Nucleopore® (Pleasanton, Calif.) membranes with pore size 0.2 μm. The external solution is treated to remove any unentrapped sorbitol. The peptide boronic acid compound Bz-Leu-Leu-boroLeu (pinacol ester) (compound of FIG. 1F) is then added to the external suspension medium and the mixture is incubated overnight at 37° C. with shaking. Any unencapsulated compound is then removed.

### EXAMPLE 3

### In Vitro Activity of Liposome-Entrapped Peptide Boronic Acid Compound

Multiple myeloma cells are grown to confluence on microtiter plates. The cells are incubated with liposomes prepared as described in Example 1 at various concentrations of peptide boronic acid compound. After a 24 hour incubation period, the cells are inspected for apoptosis. It is found that cells treated with the liposome formulation have a higher incidence of apoptosis than control cells.

### EXAMPLE 4

### In Vivo Activity of Liposome-Entrapped Peptide Boronic Acid Compound

Liposomes prepared as described in Example 1 are administered in an intravenous bolus dose to rats bearing a solid tumor. Tumor size is measured as a function of time and found to decrease for animals treated with the liposome formulation.

While a number of exemplary aspects and embodiments have been discussed above, those of skill in the art will recognize certain modifications, permutations, additions and sub-combinations thereof. It is therefore intended that the following appended claims and claims hereafter introduced are interpreted to include all such modifications, permutations, additions and sub-combinations as are within their true spirit and scope.

It is claimed:

1. A composition, comprising
liposomes formed of a vesicle-forming lipid, and
entrapped in said liposomes, a boronate ester compound prepared from a peptide boronic acid compound and a polyol, with the proviso that the peptide boronic acid compound is not bortezomib.

2. The composition of claim 1, wherein said peptide boronic acid compound is a dipeptidyl boronic acid compound.

3. The composition of claim 1, wherein said polyol is a compound having a cis 1,2-diol functionality or a 1,3-diol functionality.

4. The composition of claim 1, wherein said polyol is polyvinylalcohol.

5. The composition of claim 1, wherein said polyol is a monosaccharide, a disaccharide, an oligosaccharide, or a polysaccharide.

6. The composition of claim 5, wherein said polyol is a monosaccharide selected from maltose, glucose, ribose, fructose, and sorbitol.

7. The composition of claim 1, wherein said polyol is glycerol or polyglycerol.

8. The composition of claim 1, wherein said polyol is an aminopolyol.

9. The composition of claim 8, wherein said aminopolyol is an aminosorbitol.

10. The composition of claim 8, wherein said aminopolyol is a copolymer of vinyl alcohol and vinyl amine.

11. The composition of claim 1, wherein said liposomes further comprise a higher inside/lower outside ion gradient.

12. The composition of claim 11, wherein said ion gradient is a hydrogen ion gradient.

13. The composition of claim 12, wherein said hydrogen ion gradient provides an inside pH of between about 7.5-8.5 and an outside pH of between about 6-7.

14. The composition of claim 1, wherein said liposomes further comprise between about 1-20 mole percent of a hydrophobic moiety derivatized with a hydrophilic polymer.

15. The composition of claim 14, wherein said hydrophobic moiety derivatized with a hydrophilic polymer is a hydrophobic moiety derivatized with polyethylene glycol.

16. The composition of claim 15, wherein said hydrophobic moiety is a lipid.

17. A treatment method, comprising
preparing liposomes having in entrapped form a boronate ester compound prepared from a peptide boronic acid compound and a polyol, and;
administering the liposomes to a subject.

18. The method of claim 17, wherein said preparing further includes preparing a suspension of liposomes having a higher inside/lower outside ion gradient and adding said peptide boronic acid compound to the suspension to achieve uptake of the peptide boronic acid compound into the liposomes for formation of a peptidyl boronate ester compound.

19. The method of claim 17, wherein said administering is via injection.

20. The method of claim 17, wherein said subject has a hematologic malignancy.

21. A method of selectively destroying tumor tissue in a tumor-bearing subject undergoing radiation therapy, comprising
administering to a tumor-bearing subject, liposomes having in entrapped from (i) a boronate ester compound prepared from a peptide boronic acid compound and a polyol and (ii) an isotope of boron; and
subjecting said subject to radiation therapy.

22. The method of claim 21, wherein said isotope of boron is on the peptide boronic acid compound.

23. The method of claim 21, wherein said isotope of boron is a ¹⁰B.
